(19) United States
(12) Patent Application Publication
(10) Pub. No.: US 2025/0255573 A1
(43) Pub. Date: Aug. 14, 2025

(54) APPARATUS AND METHOD FOR CLASSIFYING AN AUDIO SIGNAL

(71) Applicants: BOEHRINGER INGELHEIM VETMEDICA GMBH, Ingelheim am Rhein (DE); FRAUNHOFER GESELLSCHAFT ZUR FÖRDERUNG DER ANGEWANDTEN FORSCHUNG E.V., München (DE)

(72) Inventors: Pinar BISGIN, Dortmund (DE); Patrick RENNINGER, Ingelheim am Rhein (DE); Christoph Matthias SCHUMMER, Ingelheim am Rhein (DE)

(21) Appl. No.: 19/116,007

(22) PCT Filed: Sep. 26, 2023
(86) PCT No.: PCT/EP2023/076547

§ 371 Date: Mar. 27, 2025

(30) Foreign Application Priority Data
Sep. 28, 2022 (EP) 22198388.5
Sep. 30, 2022 (EP) 22198974.2

(51) Int. Cl. A61B 7/04; A61B 5/00; G06F 3/16

(52) U.S. Cl. CPC A61B 7/04; A61B 5/7264; G06F 3/162; A61B 2503/40

(57) ABSTRACT

An apparatus for classifying at least one audio signal has an input interface configured to receive input information of the audio signal, a trained first machine-learning-based classifier configured to map the input information to one of a first and a second class of audio signals; a trained second machine-learning-based classifier configured to, if the audio signal belongs to the first class of audio signals, map the input information of the audio signal belonging to the first class of audio signals to one of a plurality of third classes of audio signals, and an output interface configured to output information on which classes the audio signal belongs to.

### BACKGROUND OF THE INVENTION

### Field of the Invention

The present disclosure relates to an apparatus for classifying an audio signal and to a corresponding method which may be used for determining unwanted effects, like damage to machinery or a disease of a mammal.

### Description of Related

An audio signal or acoustic signal may enable determination of unwanted effects, like damage to machinery or a disease of a mammal, such as a non-human mammal, in particular a canine, more particularly a dog.

Non-human mammals herein especially mean companion animals or pets, terms which are to be understood synonymously herein. Pet or companion animal means a domesticated animal kept for pleasure rather than utility, e.g., feline animal such as a cat, canine animal such as a dog, and horse. In particular, herein, pet means a dog.

Myxomatous mitral valve disease (MMVD, also known as endocardiosis and degenerative or chronic valvular heart disease) is a cause of prolapse of mitral valve leaflet(s) into the left atrium of the heart. Complications of myxomatous mitral valve disease include infective endocarditis, mitral regurgitation, sudden death, and stroke.

MMVD is the most common heart disease in dogs. A staging system for MMVD describes four basic stages of heart disease and heart failure: stage A, stage B (including B1 and B2), stage C, and stage D. Annually, approximately 4.3 million dogs worldwide suffer from stage B2 heart disease that is either misdiagnosed or diagnosed late. Stage B2 refers to asymptomatic dogs that have more advanced mitral valve regurgitation that is hemodynamically severe and long-standing enough to have caused radiographic and echocardiographic findings of left atrial and ventricular enlargement that meet clinical trial criteria used to identify dogs that clearly should benefit from initiating pharmacologic treatment to delay the onset of heart failure. The signs of MMVD as it progresses are coughing, increased respiratory rate, shortness of breath, listlessness, poor performance, reluctance to eat, brief periods of loss of consciousness. The causes are an irregular heartbeat, or severe coughing or as a result of a tear in the left atrium.

Prevalence increases with age, affecting about 10% of all 5- to 8-year-old dogs, about 25% of all 9- to 12-year-old dogs, and 35% of all dogs over 13 years of age. It mainly affects older dogs of small breeds (<20 kg), such as: Miniature Poodle, Mini Schnauzer, Yorkshire Terrier, Dachshund. Another predisposed dog breed is the Cavalier King Charles Spaniel. It represents a peculiarity in that it often develops mitral endocardiosis at a young age. Large dogs are, by far, less often affected.

Heart murmur as a deviation from healthy heart sound is the first and most important criterion in the diagnosis of MMVD. Heart murmur can be heard by a veterinarian with the help of a stethoscope, even before the owner himself notices any changes in his own pet. Therefore, this disease may be detected during routine examinations, such as vaccination examinations. Nevertheless, this is difficult for the general veterinarian to diagnose and requires training, experience, and expert validation. To diagnose MMVD, accurate staging of the heart murmur is a prerequisite. A distinction is made between stages B1 (without cardiac enlargement), B2 (with cardiac enlargement), and C (acute or previous cardiac failure). Beginning with stage B2, a drug (e.g., Pimobendan) can be used as a cardiovascular agent to treat the patient effectively. There is a correlation with the stages and the noise intensity of the disease. The murmur is caused by turbulent blood flow passing through the damaged leaflets of the mitral valve from the left ventricle to the left atrium. The loudness of the murmur thereby determines its “grade.” An alternative method of grading murmurs provides:
Quiet (quieter than heart sounds)=Grades I and II.
Moderate (as loud as the heart sounds)=Grade III
Loud (louder than the heart sounds)=Grade IV
Droning (very loud, heard when the stethoscope is removed from the chest)=Grades V and VI.

MMVD is usually classified as mild, moderate, or severe. While quiet mitral sounds—grade I or II—almost always indicate mild MMVD, there is often no correlation between the degree of the sound and the extent of mitral regurgitation once the sound goes up from there. Thus, as with stage B1, this is usually not treated directly with a drug (e.g. Pimobendan). Almost certainly, physicians will give the appropriate medication to patients at grade IV and above. In the case of moderate murmurs, heart ultrasound must be used to ensure that there is no enlargement. Only then can therapy be given.

Heart murmur identification is the first and also the most important criterion in diagnosis (of MMVD). The examining physician listens to the heart sounds with the help of a stethoscope. Depending on the level of experience and training in cardiology, the animal is referred to a cardiologist. The veterinary cardiologist—most probably will re-examine the heart sounds. This is usually followed by further clinical examinations such as:

### X-Ray

Heart size: First there is an enlargement of the heart shadow in the area of the left atrium and later also in the area of the left ventricle.

Displacement of the left truncal bronchus.

Another important function of the X-ray is the evaluation of the pulmonary vessels and the pulmonary field. If the pulmonary veins are congested, this is an indication for therapy. If pulmonary edema is present, alveolar shadowing can be visualized, usually in the hilar region.

Pulmonary congestion: first the pulmonary veins appear congested, later pulmonary edema (water on the lungs) can be diagnosed.

### Echocardiography

The size of the atrium and ventricle can be measured so that any enlargement can be reliably detected (stage B1 or B2).

The ability of the heart muscle to contract can be measured.

In addition, color Doppler echocardiography can be used to quantify the extent of insufficiency.

X-rays should always be performed when a murmur is predominant (ACVIM Guidelines 2019). The differences between stages B1, B2, and C are evident on radiograph/echocardiography. Stage B1 describes asymptomatic dogs that have no radiographic or echocardiographic evidence of cardiac remodeling in response to their MMVD. Only changes are observed, but they are not severe enough to warrant treatment. Stage B2 refers to asymptomatic dogs with advanced mitral valve regurgitation that is hemodynamically severe and has been present long enough to cause radiographic and echocardiographic findings of left atrial and ventricular enlargement that meet clinical trial criteria used to identify dogs that should clearly benefit from pharmacologic treatment to delay the onset of heart failure. Stage C refers to dogs with current or previous clinical signs of heart failure caused by MMVD.

There are important treatment differences between dogs with acute heart failure that require hospitalization and those in which heart failure can be treated at home.

It has been found that improvements in the analysis of an audio or acoustic signal may lead to significant improvements in the determination of damage or disease, e.g., regarding accuracy and reliability. Therefore, it is an objective of the present invention to improve automatic identification of damages or diseases based on an analysis of audio signals.

### SUMMARY OF THE INVENTION

This objective of the present invention is addressed by the subject matter of the following description.

According to a first aspect of the present disclosure, an apparatus for classifying at least one audio signal is proposed. The apparatus comprises an input interface configured to receive input information of the audio signal. The input information of the audio signal may be the audio signal itself or other characteristics thereof. The apparatus further comprises a trained first machine-learning-based classifier configured to map the input information of the audio signal to one of a first and a second class of audio signals. The apparatus further comprises a trained second machine-learning-based classifier configured to, if the audio signal belongs to the first class of audio signals, map the input information of the audio signal belonging to the first class of audio signals to one of a plurality of third classes of audio signals. The apparatus further comprises an output interface configured to output information on which classes the audio signal belongs to.

The proposed apparatus may automatically classify the audio signal into one of a plurality of classes based on the information of the audio signal. The proposed apparatus may be used to automatically identify and classify damages to machinery or heart diseases, for example.

The trained first machine-learning-based classifier uses all audio signals and examines whether they belong to the first or the second class. The predicted second class of audio signals is not used for the trained second machine-learning-based classifier. Instead, only the predicted first class of audio signals, i.e., only a subset of all audio signals used at the beginning, will enter the trained second machine-learning-based classifier. By subdividing the classification into two subsequent and distinct classification stages, computational complexity of the involved machine-learning-based classifiers may be reduced. This may also lead to better classification results.

In some embodiments, the audio signal comprises a cyclic (or quasi-periodic) audio pattern such as, e.g., a cyclic sound of a machine, a train passing a railway sleeper, or a plurality of cycles of a heart sound. The cyclic heart sound may comprise a heart murmur of a (non-human) mammal, such as a dog, for example.

In some embodiments, the apparatus further comprises a preprocessor configured to preprocess the audio signal and to generate the input information of or on the audio signal. For example, the preprocessor comprises one or more filters configured to filter the audio signal. The one or more filters may comprise low-pass, high-pass, and/or bandpass filters for removing unwanted frequency components and/or noise from the audio signal.

In some embodiments, the preprocessor is configured to extract, from the audio signal, a plurality of features characterizing the audio signal. The extracted features may have a lower dimension (lower data size) than the audio signal. The extracted features may be considered as the input information of/on the audio signal. For example, the preprocessor may be configured to extract time-domain features and/or frequency-domain features characterizing the audio signal based on time domain and/or frequency domain analysis methods. Examples of such domain analysis methods include Fourier transforms, in particular Fast-Fourier-Transforms, Power Spectral Density, or Wavelet Decomposition transforms.

Examples of extractable characteristic features are:
a maximum, a mean, median, standard deviation, variance, skewness, kurtosis, mean absolute deviation, quantile 25th, quantile 75th, entropy, zero crossing rate, crest factor, duration of a first peak and/or second peak within the pattern, duration between a first peak and a second peak within the pattern, duration between the second peak of a first pattern and the first peak of a subsequent pattern, mel frequency cepstral coefficients (MFCC), pitch chroma, spectral flatness, spectral kurtosis, spectral skewness, spectral slope, spectral entropy, dominant frequency, bandwidth, spectral centroid, spectral flux, spectral roll off, class information, severity information, position information, race information, weight information, additional information and/or other parameters or a combination thereof.

In some embodiments, the preprocessor is configured to divide the (filtered) audio signal into a plurality of time windows, each time window comprising at least one cycle of a cyclic audio pattern (e.g., a heartbeat cycle), and to extract, from each time window, the features characterizing the audio signal of the time window. The audio signal, like a series of heartbeats or a series of side sounds resulting from a rotary machine may have a periodicity. By knowing/determining this periodicity, the audio signal can be subdivided into a plurality of windows, such that each window comprises at least one of a repeated/cyclic audio pattern. This may enable to analyze each of the repeated audio patterns independently from the other, e.g., by comparing this audio pattern with a known audio pattern. Alternatively, the repeated audio pattern can be analyzed with respect to another audio pattern subsequent to the respective audio pattern.

It should be noted that the repeated audio patterns may be substantially equal to each other, similar to each other, comprise one or more peaks of a comparable shape (shape of the respective amplitude plotted over the time) and/or comprise one or more peaks of comparable shape (shape of the altitude plotted over the time) and comparable amplitude values at respective points of time within a window length, etc. According to embodiments, the window lengths may be equal. For example, the window lengths may be determined based on a frequency of the repetition of the repeated pattern. According to another variant, a border between two audio patterns may be determined so as to determine the window length for the respective time window. This means that each window length for each window may be determined separately.

In some embodiments, the first class of audio signals denotes an irregular audio signal, and the second class of audio signals denotes a regular audio signal. Here, an “irregular audio signal” may be understood as an anomalous (abnormal) audio signal. That is, an audio signal with a signal course deviating from a normal or expected signal course. To the contrary, a “regular audio signal” may denote an audio signal with a signal course corresponding to a normal or expected signal course. For example, the first class of audio signals may denote a pathological heart murmur, while the second class of audio signals may denote a healthy heart sound. In some embodiments, the first class of audio signals may be indicative of myxomatous mitral valve disease (MMVD).

In some embodiments, the plurality of third classes of audio signals relate to different irregularity levels of the audio signal. For example, the plurality of third classes of audio signals may relate to different pathological heart murmur levels. If the first class of audio signals is indicative of pathological heart murmur, for example, the plurality of third classes may relate to different severity levels of pathological heart murmur, such as mild, moderate, or loud/thrilling.

In some embodiments, the trained first machine-learning-based classifier is configured to implement a trained first boosting algorithm. The trained second machine-learning-based classifier is configured to implement a trained second boosting algorithm. In machine learning, boosting is an ensemble meta-algorithm for primarily reducing bias, and also variance in supervised learning, and a family of machine learning algorithms that convert weak learners to strong ones. Most boosting algorithms consist of iteratively learning weak classifiers with respect to a distribution and adding them to a final strong classifier. Examples for boosting algorithms are XGBoost (eXtreme Gradient Boosting) or AdaBoost (Adaptive Boosting) which is a statistical classification meta-algorithm.

In some embodiments, the trained first and second machine-learning-based classifier are of the same type (same model or algorithm) and differ by different training signals und by different model parameters. The first machine-learning-based classifier may be trained based on first ground truth audio signals comprising the first class and the second class of audio signals to enable the first machine-learning-based classifier to determine if the audio signal belongs to the first or to the second class of audio signals. It is known beforehand which of the first ground truth audio signals are related to which of the first and second classes of audio signals. The second machine-learning-based classifier may be trained based on second ground truth audio signals comprising the first class but not the second class of audio signals to enable the second machine-learning-based classifier to determine if the audio signal belongs to one of the plurality of third classes. It is known beforehand which of the second ground truth audio signals are related to which of the third classes of audio signals. For example, training signals for the first machine-learning-based classifier may include known (features of) audio signals of both pathological and healthy heart sound. Training signals for the second machine-learning-based classifier may only include known (features of) audio signals of pathological heart murmur.

In some embodiments, the apparatus may further comprise a trained third machine-learning-based classifier configured to, if the audio signal belongs to one of the third classes of audio signals and fulfills an additional criterion, map the information of an audio signal belonging to the first class and belonging to one of the plurality of third classes to one of a plurality of fourth classes of audio signals. The additional criterion may be based on (or include) an age and/or a breed of a mammal (e.g., a dog) the audio signal belongs to. The plurality of fourth classes may correspond to a staging system for MMVD. The staging system may include stage A, stage B (B1, B2), stage C, and stage D. In particular, the plurality of fourth classes may include stages B1 and B2 for MMVD.

In some embodiments, the trained first, second, and third second machine-learning-based classifiers are of the same type (e.g., boosting algorithm) and differ by different respective training signals and model parameters. The third machine-learning-based classifier may be trained based on third ground truth audio signals of the first class but not the second class of audio signals and fulfilling an additional criterion to enable the third machine-learning-based classifier to determine if the audio signal belongs to one of the plurality of fourth classes. It is known beforehand which of the third ground truth audio signals are related to which of the fourth classes of audio signals. The additional criterion may be based on (or include) an age and/or a breed of a mammal (e.g., a dog) the audio signal belongs to. That is, for training the third machine-learning-based classifier, only third ground truth audio signals corresponding to a certain age and/or breed and belonging to first class but not the second class may be used.

In some embodiments, the input interface and/or the output interface is configured as a wireless interface. This may enable convenient transfer of audio signals and/or results to or from the apparatus. For example, audio signals by be sent to an app running on a smartphone or another portable device implementing the apparatus for classifying the audio signal.

According to a further aspect of the present disclosure, it is proposed a method for classifying an audio signal. The method includes
receiving input information of at least one audio signal;
classifying, by a first machine-learning-based classifier, the audio signal into one of a first and a second class of audio signals based on the input information of the audio signal;
if the audio signal belongs to the first class of audio signals,
classifying, by a second machine-learning-based classifier, the audio signal into one of a plurality of third classes of audio signals based on the input information of the audio signal; and
outputting information on which classes the audio signal belongs to.

In some embodiments, the method further includes, during a training phase, training the first machine-learning-based classifier by means of ground truth audio signals comprising the first class and second class of audio signals, to enable the first machine-learning-based classifier to determine if the audio signal belongs to the first or to the second class of audio signals, and training the second machine-learning-based classifier by means of ground truth audio signals comprising the first class but not the second class of audio signals, to enable the second machine-learning-based classifier to determine if the audio signal belongs to one of the plurality of third classes, wherein it is known beforehand which of the ground truth audio signals are related to which of the third classes of audio signals.

In some embodiments, the method includes classifying, by a third machine-learning-based classifier, the audio signal into one of a plurality of fourth classes of audio signals based on the input information of the audio signal if the audio signal belongs to one of the third classes of audio signals and fulfills an additional criterion.

The third machine-learning-based classifier may be trained based on third ground truth audio signals of the first class but not the second class of audio signals and fulfilling the additional criterion to enable the third machine-learning-based classifier to determine if the audio signal belongs to one of the plurality of fourth classes. It is known beforehand which of the third ground truth audio signals are related to which of the fourth classes of audio signals. The additional criterion may be based on (or include) an age and/or a breed of a mammal (e.g., a dog) the audio signal belongs to. That is, for training the third machine-learning-based classifier, only third ground truth audio signals corresponding to a certain age and/or breed and belonging to first class but not the second class may be used.

In some embodiments, the audio signal comprises cyclic heart sounds, wherein the first class of audio signals denotes a pathological heart murmur, and the second class of audio signals denotes a healthy heart sound, wherein the plurality of third classes of audio signals relate to different pathological heart murmur severity levels.

According to a further aspect of the present disclosure, it is proposed a computer program having a program code for performing the method above, when the computer program is executed on a computer, a processor, or a programmable hardware component.

As indicated above, a possible application of embodiments of the present disclosure is the diagnosis of a disease for an animal, such as a non-human mammal, in particular a dog. Therefore, according to embodiments, the audio signal may be a record of a heartbeat sequence of a dog or another animal or another non-human mammal, and/or a record of a heart murmur sequence of a dog or another animal or another non-human mammal.

Some examples of apparatuses and/or methods will be described in the following by way of example only, and with reference to the accompanying figures.

### BRIEF DESCRIPTION OF THE DRAWINGS

FIG. 1 illustrates a schematic block diagram of an apparatus for classifying at least one audio signal in accordance with the present invention;

FIG. 2 illustrates an example of an audio signal corresponding to a cyclic heart sound;

FIG. 3 shows a flowchart of a method for classifying at least one audio signal in accordance with the present invention; and

FIG. 4 shows a flowchart of a method for classifying at least one audio signal in accordance with an embodiment of the present invention.

### DETAILED DESCRIPTION OF THE INVENTION

Some examples are now described in more detail with reference to the accompanying figures. However, other possible examples are not limited to the features of these embodiments described in detail. Other examples may include modifications of the features as well as equivalents and alternatives to the features. Furthermore, the terminology used herein to describe certain examples should not be restrictive of further possible examples.

Throughout the description of the figures, same or similar reference numerals refer to same or similar elements and/or features, which may be identical or implemented in a modified form while providing the same or a similar function. The thickness of lines, layers and/or areas in the figures may also be exaggerated for clarification.

When two elements A and B are combined using an “or”, this is to be understood as disclosing all possible combinations, i.e., only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a”, “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not exclude the presence or addition of one or more other features, integers, steps, operations, processes, elements, components and/or a group thereof.

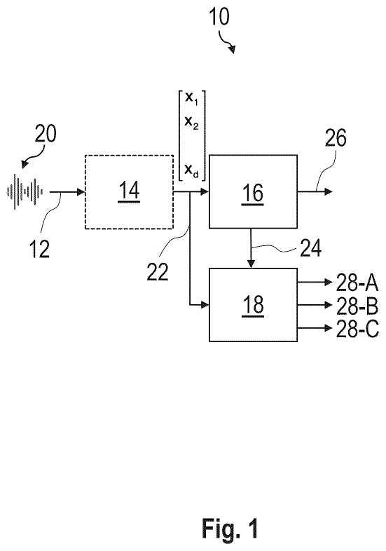

FIG. 1 schematically shows a block diagram of an apparatus 10 for classifying an audio signal. The apparatus 10 may be a programmable hardware device including memory and one or more processing units, such as CPUs and/or GPUs. The apparatus 10 may be a mobile phone, a tablet, a personal computer or the like. In other embodiments, the apparatus 10 may be implemented by a central server.

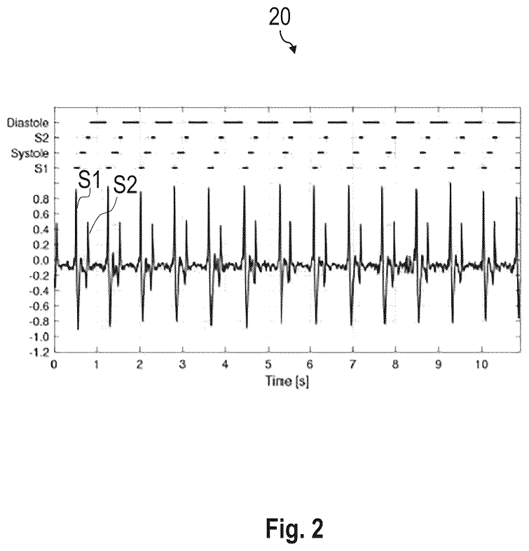

The apparatus 10 comprises an input interface 12 which is configured to receive at least one audio signal 20 or other characteristic information thereof. The audio signal 20 may be a digital representation of an acoustic signal. For example, the audio signal 20 may be received in Waveform Audio File Format (wav file). The audio signal 20 may, for example, be associated with a heartbeat of a mammal's (e.g., a dog's) heart and may thus include a cyclic or quasi-periodic audio pattern, such as a plurality of cycles of a cyclic heart sound. Heart murmurs are unique heart sounds produced when blood flows across a heart valve or blood vessel. An example of an audio signal 20 corresponding to a cyclic heart sound is shown in FIG. 2. For example, one cycle of heart sound may be defined as the duration from signal (peak) S1 to the next subsequent signal (peak) S1. Subsequent cycles of a heart sound may typically be slightly different from each other in duration and/or the amplitude of signal peaks S1, S2. The skilled person having benefit from the present disclosure will appreciate that—without departing from the principles proposed herein—the audio signal 20 may also be associated with other acoustic signals, such as acoustic signals stemming from machinery, for example.

The input interface 12 of apparatus 10 may be a wired or wireless interface, such as a Universal Serial Bus (USB), a WiFi interface, a Bluetooth interface, an infrared interface, or a cellular communications interface. In this way, the audio signal 20 (or other characteristic information thereof) can be conveniently transferred from a data source to the apparatus 10.

The apparatus 10 may optionally comprise a preprocessor 14 which may be configured to filter the audio signal 20 in order to remove or reduce undesired signal components, such as noise, for example. Therefore, the preprocessor 14 may comprise one or more digital filters, such as lowpass, bandpass, or high pass filters.

Additionally, or alternatively, the optional preprocessor 14 may be configured to extract, from the audio signal 20, a plurality of features 22 characterizing the audio signal 20. The preprocessor 14 may be configured to extract time-domain features and/or frequency-domain features from the audio signal 20, wherein the time-domain and/or frequency-domain features characterize or identify the audio signal 20. Examples of domain analysis methods include Fourier transforms, in particular Fast-Fourier-Transforms, Power Spectral Density, or Wavelet Decomposition transforms. Feature extraction starts from an initial set of measured data (audio signal 20) and builds derived values (features) intended to be informative and non-redundant, facilitating subsequent learning and generalization steps, and in some cases leading to better human interpretations. Feature extraction is related to dimensionality reduction. Thus, a dimension of the extracted plurality of features 22 is smaller than a dimension of the audio signal 20. The plurality of features 22 may be also considered as feature vector containing characteristic information of the audio signal.

The preprocessor 14 may be configured to automatically extract characteristic features of the audio signal 20, such a mammal's heart sounds. For this purpose, the audio signal 20 may be decomposed in a plurality of time windows, each time window comprising a heartbeat cycle. Therefore, the feature extraction processor 14 may be configured to divide the audio signal 20 into a plurality of time windows. For example, a determination of a time window may be based on an algorithm finding repetitions within the audio signal 20. Each time window may comprise at least one cycle of a cyclic audio pattern such as a heartbeat cycle, for example. The feature extraction processor 14 may be configured to extract, from each time window, the features 22 characterizing the audio signal of the time window.

Window lengths may be determined based on the duration of the audio signal 20 and the number of cycles of a cyclic audio pattern. The calculation may be performed by a simple division. Of course, the window lengths may, according to further embodiments, be determined differently, e.g., by determining the duration of each cycle, e.g., the interval between a systole (diastole) and the subsequent systole (diastole) and averaging these durations. According to further embodiments, the window lengths may vary over time, e.g., when the periodicity of the pattern varies. This can happen, e.g., when the heartbeat rate decreases in the current situation.

Possible time domain features which can be extracted from the audio signal 20 or its windows are:

The mean of the audio pattern, the median of the audio pattern, the standard deviation of the audio pattern, the variance of the audio pattern or with respect to another pattern, the skewness of the audio pattern, the kurtosis of the audio pattern, the mean absolute deviation of the audio pattern, the quantile 25th of the audio pattern, the quantile 75th of the audio pattern, the entropy of the audio pattern, the zero-crossing rate of the audio pattern, the quest factor, the duration of a first peak, the duration of another peak, the duration from the end of S1 to the start of the next S1, the duration of end of S2 to the start of the next S1. Especially, the duration features are more meaningful when the audio signal 20 is divided into windows.

Possible frequency domain features which can be extracted from the audio signal 20 or its windows are:

The mel frequency cepstral coefficients, the pitch chroma, the spectral flatness, the spectral kurtosis, the spectral skewness, the spectral slope, the spectral entropy, the dominant frequency, the bandwidth, the spectral centroid, the spectral flux, and/or the spectral roll off.

It should be noted that the lists for the different feature types and the feature type is not limited to the mentioned ones. According to embodiments, the feature extraction may be mainly or completely performed automatically. Especially, the windowing may be performed automatically.

The apparatus 10 further comprises a trained first machine-learning-based classifier 16 configured to map information of the audio signal 20 to one of a first and a second class of audio signals 24, 26. The information of the audio signal may, in one embodiment, correspond to the optionally extracted features 22 or, in another embodiment, to the audio signal 20 itself. In other words, the trained first machine-learning-based classifier 16 is configured to decide whether the audio signal 20 or the extracted features 22 thereof are indicative of the first class 24 of audio signals or the second class 26 of audio signals.

In some embodiments, the first class 24 of audio signals denotes an irregular audio signal, and the second class 26 of audio signals denotes a regular audio signal. Here, an “irregular audio signal” may be understood as an abnormal audio signal. That is, an audio signal with a signal course deviating from a normal or expected signal course. To the contrary, a “regular audio signal” may denote an audio signal with a signal course corresponding to a normal or expected signal course. In the example related to audio signals 20 including heart sounds, the first class 24 of audio signals may be indicative of a pathological heart murmur, while the second class 26 of audio signals may be indicative of a healthy heart sound. Thus, the first class of audio 24 signals may be indicative of myxomatous mitral valve disease (MMVD) while the second class 26 of audio signals may be indicative of a healthy (non-human) mammal (e.g., a dog).

The trained first machine-learning-based classifier 16 may, for instance, be based on a neural network, a random forest algorithm or a boosting algorithm, in particular on an XGboost algorithm.

The apparatus 10 further comprises a trained second machine-learning-based classifier 18 configured to map the extracted features 22 or the audio signal 20 belonging to the first class 24 of audio signals to one of a plurality of third classes 28A-C of audio signals. Thus, the trained second machine-learning-based classifier 18 maps the extracted features 22 or the audio signal belonging to the first class 24 to either class 28-A, class 28-B, or class 28-C. The skilled person having benefit from the present disclosure will appreciate that a different number (or amount) of third classes 28 is also possible. The trained second machine-learning-based classifier 18 is used only for audio signals 20 (or characteristic features thereof) belonging to the first class 24 of audio signals (e.g., pathological heart murmur) and not for audio signals 20 (or characteristic features thereof) belonging to the second class 26 of audio signals (e.g., healthy heart sound).

The plurality of third classes 28A-C of audio signals may relate to different irregularity levels of the audio signal 20. That is, the plurality of third classes 28A-C of audio signals may be indicative of how much the audio signal 20 deviates from a normal or expected audio signal (e.g., healthy heart sound). For example, the plurality of third classes 28A-C of audio signals may relate to different pathological heart murmur levels. If the first class 24 of audio signals is indicative of pathological heart murmur or MMVD, for example, the plurality of third classes 28-A-C may relate to different severity levels of pathological heart murmur, such as mild, moderate, or loud/thrilling. Thus, the output of the trained second machine-learning-based classifier 18 may indicate whether the audio signal 20 contains mild pathological heart murmur (class 28-A, mild deviation from healthy heart sound), moderate pathological heart murmur (class 28-B, moderate deviation from healthy heart sound), or loud/thrilling pathological heart murmur (class 28-C, strong deviation from healthy heart sound).

Like the trained first machine-learning-based classifier 16, the trained second machine-learning-based classifier 18 may, for instance, be based on a neural network, a random forest algorithm or a boosting algorithm, in particular on an XGboost algorithm. In particular, the trained first and second machine-learning-based classifiers 16, 18 may rely on the same type of machine-learning algorithm or model. For example, the trained first and second machine-learning-based classifiers 16, 18 may both rely on the same boosting algorithm and be programmed using the Python programming language.

However, the trained first and second machine-learning-based classifiers 16, 18 may be trained with different training signals (ground truth data). During an initial training phase prior to inference phase, the first machine-learning-based classifier 16 may be trained by means of ground truth audio signals comprising both the first class 24 (e.g., pathological heart murmur) and second class 26 (e.g., healthy heart sound) of audio signals to enable the first machine-learning-based classifier 16 to determine if the audio signal 20 belongs to the first class 24 or to the second class 26 of audio signals. Typical for ground truth data, it is known beforehand which of the ground truth audio signals (or characteristic information thereof) for the first machine-learning-based classifier 16 are related to which of the first and second classes 24, 26 of audio signals. Instead, the second machine-learning-based classifier 18 may be trained by means of ground truth audio signals comprising only the first class 24 (e.g., pathological heart murmur) but not the second class of audio signals (e.g., healthy heart sound), to enable the second machine-learning-based classifier 18 to determine if the audio signal 20 belongs to one of the plurality of third classes 28A-C. Typical for ground truth data, it is known beforehand which of the ground truth audio signals for the second machine-learning-based classifier 18 (first class audio signals) are related to which of the third classes 28A-C of audio signals.

Due to the different training data, the trained first machine-learning-based classifier 16 is configured differently from the trained second machine-learning-based classifier 18. In particular, the trained first machine-learning-based classifier 16 and the trained second machine-learning-based classifier 18 may be differently trained XGboost classifiers. XGboost classifiers are examples of classifiers based on boosting algorithms.

The apparatus 10 further comprises an output interface (not shown) configured to output information on which of classes 24, 26, 28A-C the audio signal 20 belongs to. The output interface may comprise a display and/or a wired or wireless interface, such a Universal Serial Bus (USB), a WiFi interface, a Bluetooth interface, an infrared interface, or a cellular communications interface, for example. In this way, the output information can be conveniently transferred from the apparatus 10 to a data recipient.

The skilled person having benefit from the present disclosure will appreciate that the apparatus cannot only be used to automatically classify a single audio signal, but to automatically classify a plurality of audio signals. Depending on the implementation, the plurality of audio signals may be classified subsequently or in parallel.

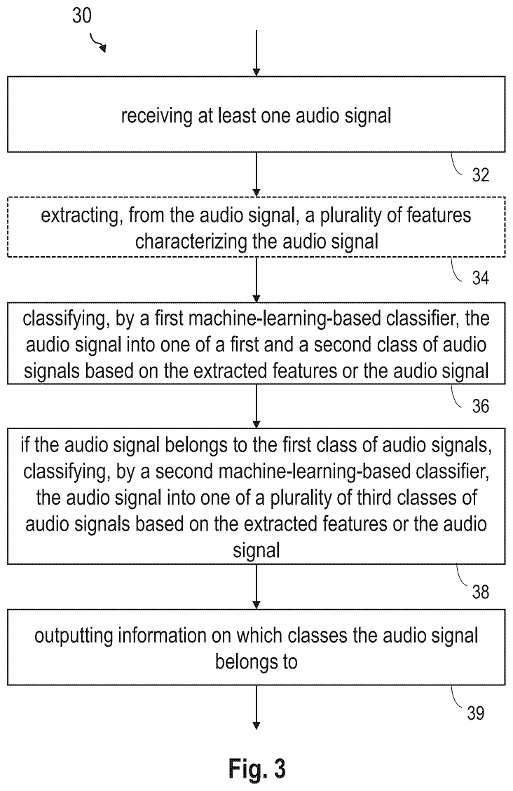

The apparatus 10 may be implemented by a single programmable hardware device or by a plurality of programmable hardware devices. Thus, the apparatus 10 is configured to carry out a computer-implemented method 30 for classifying an audio signal. A flowchart of the computer-implemented method 30 is depicted in FIG. 3.

Method 30 includes an act 32 of receiving at least one audio signal 20. Audio signal 20 may be associated with a (non-human) mammal's (e.g., a dog's) heartbeat. Further, method 30 may include an optional act 34 of (automatically) extracting, from the audio signal 20, a plurality of features 22 characterizing the audio signal 20. As mentioned before, the features 22 may include time-and/or frequency domain features characterizing the audio signal. Method 30 further includes an act 36 of classifying, by a first machine-learning-based classifier 16, the audio signal 20 into one of a first and a second class of audio signals based on the audio signal 20 or the extracted features 22. The first class 24 may relate to pathological heart murmur, the second class 26 may relate to healthy heart sound. The skilled person having benefit from the present disclosure will appreciate that the first class may as well relate to other abnormal sounds (e.g., abnormal machinery sound), while the second class 26 may as well relate to other normal sounds (e.g., normal machinery sound). Only if the audio signal 20 belongs to the first class 24 of audio signals, method 30 proceeds to an act 38 of classifying, by a second machine-learning-based classifier 18, the audio signal 20 into one of a plurality of third classes 28 of audio signals based on the extracted features 22 or the audio signal 20 itself. The third classes 28 may relate to different severity levels of pathological heart murmur. The skilled person having benefit from the present disclosure will appreciate that third classes 28 may as well relate to different severity levels of other abnormal sounds (e.g., abnormal machinery sound). Yet further, method 30 includes an act 39 of outputting information on which classes the audio signal 20 belongs to.

The method 30 may also include a further act of classifying, by a third machine-learning-based classifier, the audio signal into one of a plurality of fourth classes of audio signals. The classification may be based on the audio signal or features thereof and may only be performed if the audio signal belongs to one of the third classes of audio signals and fulfills an additional criterion. An embodiment with three sequential machine-learning-based classifiers will be described below.

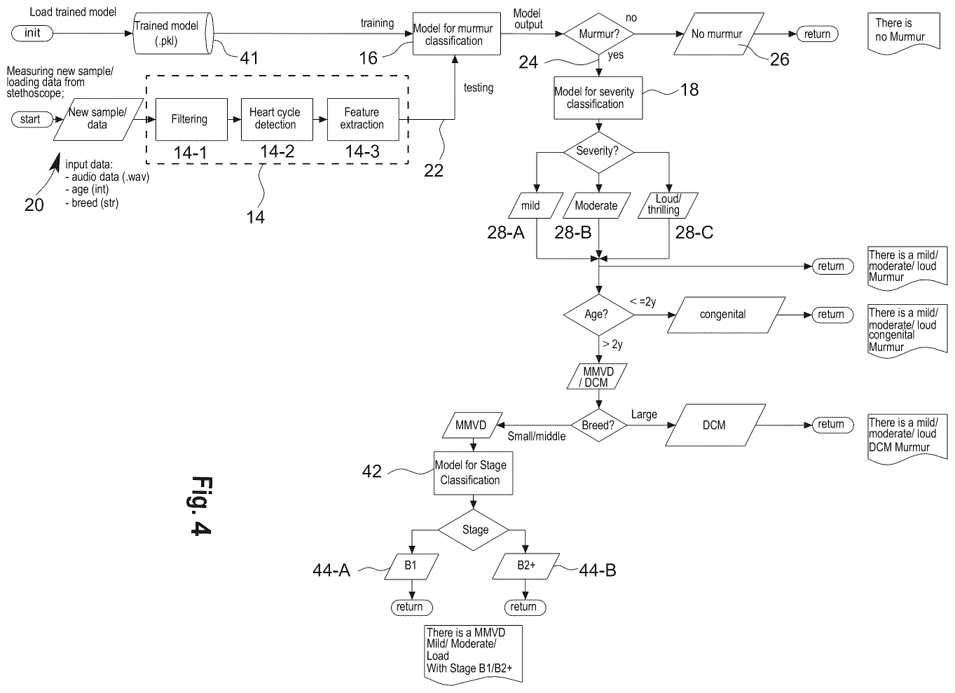

Turning now to FIG. 4, it shows a detailed flowchart of a computer-implemented method 40 for classifying an audio signal 20. Method 40 may run on a smartphone or a tablet in the form of an app, for example.

Initially, previously trained first model parameters 41 are provided for the first machine-learning-based classifier 16 (e.g., boosting algorithm), the second machine-learning-based classifier 18 (e.g., boosting algorithm), and a third machine-learning-based classifier 42 (e.g., boosting algorithm). This may be done in the form of one or more pickle files which can be used to save a machine-learning model and to serialize Python object structures. Once the first machine-learning-based classifiers 16, 18, 42 are initialized, at least one audio file containing an audio signal 20 may be provided. The audio signal 20 may be indicative of a dog's (or other mammal's) heart sound recorded by means of a stethoscope, for example. The one or more audio files 20 may, for example, come as. wav files and may be provided via a wireless interface. In addition to the audio file(s), information on an age (integer) and on a breed (string) of the dog from which the heart sound stems may be provided via the wireless interface.

The audio signal 20 extracted from the audio file may be provided to the preprocessor 14. Preprocessor 14 may comprise a bandpass filter 14-1, a heart-cycle detection unit 14-2, and a feature extraction unit 14-3. Bandpass filter 14-1 may have a passband from 50-500 Hz, for example. Further, the initial 0.5 s and the last 0.5 s of the audio signal 20 may be cut off to eliminate unwanted acoustic signals. Heart-cycle detection unit 14-2 is configured to perform the previously described windowing to obtain one or more time windows of the audio signal 20, wherein a time window may correspond to at least one heartbeat cycle of the heart sound. Feature extraction unit 14-3 is configured to extract a plurality of characteristic features 22 from the one or more windows of the audio signal 20.

The characteristic features 22 may then be provided as input to the first machine-learning-based classifier 16, which is configured to map its input to a first model output indicative of the first class 24 or the second class 26 of audio signals. That is, depending on the input features 22, the first machine-learning-based classifier 16 will predict whether the audio signal 20 is indicative of a pathological heart murmur or a healthy heart sound. If the audio signal 20 is indicative of a healthy heart sound (class 26), this information may be output and the method 40 ends or returns. A new audio signal may then be analyzed, for example.

If, on the other hand, the audio signal 20 is indicative of a pathological heart murmur (class 24), the extracted characteristic features 22 of audio signal 20 may be provided as input to the second machine-learning-based classifier 18 which is configured to map its input to a second model output indicative of one of three classes 28-A, 28-B, and 28-C of audio signals. The three classes 28-A, 28-B, and 28-C are indicative of mild, moderate, and loud/thrilling pathological heart murmur. That is, depending on the input features 22, the second machine-learning-based classifier 18 will predict whether the audio signal 20 is indicative of mild, moderate, and loud/thrilling pathological heart murmur. The respective information may be output and the method 40 may end or return.

If the dog's age is lower than or equal to a predefined age threshold (here: 2 years), the pathological heart murmur may be classified as mild, moderate, and loud/thrilling congenital heart murmur. Congenital means that the dog was born with the condition. If the dog's age is larger than the predefined age threshold (here: 2 years), this is classified as either myxomatous mitral valve disease (MMVD) or dilated cardiomyopathy (DCM). The respective information may be output and the method 40 may end or return.

If the dog's breed indicates a small or middle size dog, the pathological heart murmur is classified as mild, moderate, and loud/thrilling MMVD murmur. If the dog's breed indicates a large size dog, the pathological heart murmur is classified as mild, moderate, and loud/thrilling DCM murmur. The respective information may be output and the method 40 may end or return.

By means of the trained third machine-learning-based classifier 42 (e.g., boosting algorithm) the audio signal 20 classified as one of mild, moderate, and loud/thrilling MMVD murmur is classified into one of two MMVD stages of heart disease. Here, the two stages are B1 and B2. Stage B1 describes asymptomatic dogs that have no radiographic or echocardiographic evidence of cardiac remodeling in response to their MMVD, as well as those in which remodeling changes are present, but not severe enough to meet current clinical trial criteria that have been used to determine that initiating treatment is warranted. Stage B2 refers to asymptomatic dogs that have more advanced mitral valve regurgitation that is hemodynamically severe and long-standing enough to have caused radiographic and echocardiographic findings of left atrial and ventricular enlargement that meet clinical trial criteria used to identify dogs that clearly should benefit from initiating pharmacologic treatment to delay the onset of heart failure.

If the audio signal 20 is indicative of a pathological heart murmur (class 24), one of mild, moderate, and loud/thrilling pathological heart murmur (one of three classes 28-A, 28-B, and 28-C), the dog is older than 2 years, and its breed is small/middle, then the extracted characteristic features 22 of audio signal 20 may be provided as input to the third machine-learning-based classifier 42 which is configured to map its input to a third model output indicative of one of two classes 44-A, 44-B. The two classes 44-A, 44-B are indicative of mild, moderate, and loud/thrilling MMVD murmur with stage B1 or with stage B2. That is, depending on the input features 22, the second machine-learning-based classifier 18 will predict whether the audio signal 20 is indicative of mild, moderate, and loud/thrilling MMVD murmur with stage B1 or stage B2. The respective information may be output and the method 40 may end or return.

It is noted that the third machine-learning-based classifier 42 may be trained based on third ground truth audio signals of the first class 24 (pathological heart murmur) but not the second class and fulfilling the additional criterion dog's age>2 and dog's breed=small/middle. That is, for training the third machine-learning-based classifier 42, only third ground truth audio signals corresponding to a certain age and/or breed and belonging to first class 24 but not the second class may be used.

Embodiments of the present disclosure propose a concept that can detect intensity in addition to sound detection. For this purpose, a multi-part/cascading algorithm is proposed: A trained model, here a feature matrix, may be loaded into the algorithm in the form of a pickle file. Each new sample (audio signal) to be tested goes through the same steps as the trained model. The new sample/sounds may be filtered from noise (ambient noise, . . . ). For this purpose, different methods of time-frequency analysis may be performed (Fast Fourier Transform, Power Spectral Density, Wavelet Decomposition). Afterwards, the heart sounds may be detected by using different methods, so that several windows with one heartbeat cycle each may be obtained. Various features 22 may be calculated for the individual heart beat cycles (windows) in both the time and frequency domains. The same steps were previously performed with the pickle file with multiple data. Finally, the new test data set can be given to the first classifier 16. This uses all the data and examines whether this is a pathological heart murmur or a healthy heart sound. The predicted healthy data is not used for the second classifier 18. It follows that only the pathological data set, i.e., only a subset of the data set used at the beginning will enter the second classifier 18. This classifier 18 then examines the loudness of the heart murmur and returns whether it is a mild, moderate or loud murmur. With this information, a recommendation can be issued to a physician. Dogs under 2 years of age are likely to have a congenital murmur present. Dogs older than 2 years of age with a small/medium breed may have MMVD disease present. Large dogs usually have DCM (dilated cardiomyopathy). Boosting algorithms such as the XGBoost, AdaBoost, etc. are particularly well suited for this.

The aspects and features described in relation to a particular one of the previous examples may also be combined with one or more of the further examples to replace an identical or similar feature of that further example or to additionally introduce the features into the further example.

Examples may further be or relate to a (computer) program including a program code to execute one or more of the above methods when the program is executed on a computer, processor or other programmable hardware component. Thus, steps, operations or processes of different ones of the methods described above may also be executed by programmed computers, processors or other programmable hardware components. Examples may also cover program storage devices, such as digital data storage media, which are machine-, processor- or computer-readable and encode and/or contain machine-executable, processor-executable or computer-executable programs and instructions. Program storage devices may include or be digital storage devices, magnetic storage media such as magnetic disks and magnetic tapes, hard disk drives, or optically readable digital data storage media, for example. Other examples may also include computers, processors, control units, (field) programmable logic arrays ((F)PLAs), (field) programmable gate arrays ((F)PGAs), graphics processor units (GPU), application-specific integrated circuits (ASICs), integrated circuits (ICs) or system-on-a-chip (SoCs) systems programmed to execute the steps of the methods described above.

It is further understood that the disclosure of several steps, processes, operations or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process or operation may include and/or be broken up into several sub-steps, -functions, -processes or -operations.

If some aspects have been described in relation to a device or system, these aspects should also be understood as a description of the corresponding method. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

The following claims are hereby incorporated in the detailed description, wherein each claim may stand on its own as a separate example. It should also be noted that although in the claims a dependent claim refers to a particular combination with one or more other claims, other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

1. An apparatus (10) for classifying at least one audio signal (20), the apparatus (10) comprising
an input interface (12) configured to receive input information (22) of the audio signal (20);
a trained first machine-learning-based classifier (16) configured to map the input information (22) of the audio signal to one of a first and a second class of audio signals (24; 26);
a trained second machine-learning-based classifier (18) configured to, if the audio signal (20) belongs to the first class of audio signals (24), map the input information (22) of the audio signal belonging to the first class of audio signals (24) to one of a plurality of third classes (28) of audio signals; and
an output interface configured to output information on which classes the audio signal (20) belongs to.

2. The apparatus (10) of claim 1, wherein the audio signal (20) comprises a plurality of cycles of a heart sound.

3. The apparatus (10) of claim 2, wherein the first class (24) of audio signals denotes a pathological heart murmur, and the second class (26) of audio signals denotes a healthy heart sound.

4. The apparatus (10) of claim 2, wherein the plurality of third classes (28) of audio signals relate to different pathological heart murmur levels.

5. The apparatus (10) of claim 1, comprising a preprocessor (14) configured to extract, from the audio signal (20), a plurality of features (22) characterizing the audio signal as the input information (22) of the audio signal (20).

6. The apparatus (10) of claim 5, wherein the preprocessor (14) is configured to extract time-domain features and/or frequency-domain features characterizing the audio signal (20).

7. The apparatus (10) of claim 1, wherein the trained first machine-learning-based classifier (16) is configured to implement a trained first boosting algorithm and/or wherein the trained second machine-learning-based classifier (18) is configured to implement a trained second boosting algorithm.

8. The apparatus (10) of claim 1, wherein the trained first and second machine-learning-based classifier (16; 18) are of the same type and differ by different respective training signals.

9. The apparatus (10) of claim 8, wherein the first machine-learning-based classifier (16) is trained based on first ground truth audio signals comprising the first class (24) and second class (26) of audio signals to enable the first machine-learning-based classifier (16) to determine if the audio signal (20) belongs to the first or to the second class of audio signals, wherein it is known beforehand which of the first ground truth audio signals are related to which of the first and second classes of audio signals, and wherein the second machine-learning-based classifier (18) is trained based on second ground truth audio signals comprising the first class (24) but not the second class of audio signals to enable the second machine-learning-based classifier (18) to determine if the audio signal (20) belongs to one of the plurality of third classes (28), wherein it is known beforehand which of the second ground truth audio signals are related to which of the third classes of audio signals.

10. The apparatus (10) of claim 1, further comprising a trained third machine-learning-based classifier (42) configured to map the input information (22) of an audio signal belonging to any one of the plurality of third classes (28) of audio signals and fulfilling an additional criterion to one of a plurality of fourth classes of audio signals.

11. The apparatus (10) of claim 10, wherein the trained first, second, and third second machine-learning-based classifiers (16; 18; 42) are of the same type and differ by different respective training signals.

12. The apparatus (10) of claim 10 wherein the third machine-learning-based classifier (42) is trained based on third ground truth audio signals of the first class (24) but not the second class of audio signals and fulfilling the additional criterion to enable the third machine-learning-based classifier (42) to determine if the audio signal (20) belongs to one of the plurality of fourth classes (28), wherein it is known beforehand which of the third ground truth audio signals are related to which of the fourth classes of audio signals.

13. The apparatus (10) of claim 10, wherein the additional criterion is based on an age and/or a breed of a mammal the audio signal (20) belongs to.

14. A method for classifying at least one audio signal, the method comprising
receiving input information (22) of the audio signal (20);
classifying, by a first machine-learning-based classifier (16), the audio signal (20) into one of a first and a second class (24; 26) of audio signals based on the input information (22);
if the audio signal (20) belongs to the first class (24) of audio signals, classifying, by a second machine-learning-based classifier (18), the audio signal (20) into one of a plurality of third classes (28) of audio signals based on the input information (22) of the audio signal; and
outputting information on which classes the audio signal (20) belongs to.

15. The method of claim 14, further comprising, during a training phase,
training the first machine-learning-based classifier (16) by means of ground truth audio signals comprising the first class (24) and second class (26) of audio signals, to enable the first machine-learning-based classifier (16) to determine if the audio signal (20) belongs to the first or to the second class of audio signals; and
training the second machine-learning-based classifier (18) by means of ground truth audio signals comprising the first class (24) but not the second class of audio signals, to enable the second machine-learning-based classifier (18) to determine if the audio signal belongs to one of the plurality of third classes (28), wherein it is known beforehand which of the ground truth audio signals are related to which of the third classes of audio signals.
